(19) United States
(12) Patent Application Publication
(10) Pub. No.: US 2013/0157877 A1
(43) Pub. Date: Jun. 20, 2013

(54) BIOCHIPS FOR ANALYZING NUCLEIC ACID MOLECULE DYNAMICS

(71) Applicants: Thomas Plenat, Toulouse (FR); Laurence Salome, Auzeville Tolosane (FR); Catherine Tardin, Toulouse (FR); Christophe Thibault, Toulouse (FR); Emmanuelle Trevisiol, Montcabrier (FR); Christophe Vieu, Auzeville Tolosane (FR)

(72) Inventors: Thomas Plenat, Toulouse (FR); Laurence Salome, Auzeville Tolosane (FR); Catherine Tardin, Toulouse (FR); Christophe Thibault, Toulouse (FR); Emmanuelle Trevisiol, Montcabrier (FR); Christophe Vieu, Auzeville Tolosane (FR)

(73) Assignees: Institut National Des Sciences Appliquees De Toulouse, Toulouse (FR); Centre National De La Recherche Scientifique-Cnrs, Paris (FR)

(21) Appl. No.: 13/819,664

(22) PCT Filed: Sep. 1, 2011
(86) PCT No.: PCT/FR2011/052004

§ 371 Date: Feb. 27, 2013

(30) Foreign Application Priority Data
Sep. 3, 2010 (FR) 1057031

(51) Int. Cl. C12Q 1/68

(52) U.S. Cl. CPC C12Q 1/6876
USPC 506/7; 506/18; 506/30; 506/16

(57) ABSTRACT

The invention relates to biochips 1 comprising a substrate 2, wherein said substrate comprises at the surface thereof isolated regions 3 for the anchoring of a nucleic acid molecule, said isolated regions having an area of less than 1 μm², and the space 4 between two isolated regions being at least equal to the square root of the value of said area of said isolated regions.

### FIELD OF THE INVENTION

The invention relates to the field of biochips for analyzing nucleic acid molecule dynamics.

### INTRODUCTION

Many techniques have been developed for studying nucleic acid molecule dynamics. Some of them consist in attaching a bead to a surface via a nucleic acid molecule. Forces can then be applied to the beads, in particular by means of a magnet, of a flow of liquid, or of electrostatic repulsion. In the simplest case, corresponding to the “tethered particle motion” or “TPM” technique, a bead is attached to the surface of a coverslip via a single nucleic acid molecule and undergoes Brownian motion in the absence of applied external force. The movements and/or the trajectories are then observed, for example under an optical microscope, in order to evaluate the size of the motion of the bead, which is directly dependent on the length of the nucleic acid molecule. This technique was described for the first time by Schafer et al., in 1991 (Nature, 352, 444-448).

This approach has been successfully applied to the study of the elongation of transcripts produced by an RNA polymerase (Yin et al., 1994, Biophys. J., 67, 2468-2478), to the analysis of the kinetics of loop formation on DNA by the lactose repressor protein (Finzi et al., 1995, Science, 267, 378-380), or else to the study of DNA translocations by the RecBCD enzyme (Dohoney et al., 2001, Nature, 409, 370-374).

However, the TPM technique has technical limitations, the anchoring of the nucleic acids is generally not stable since the anchoring molecules are simply adsorbed onto the substrate and the density of bead/DNA complexes bound to the substrate is low so as to limit the probability of neighboring beads influencing one another. Thus, it does not make it possible to analyze a very large number of molecules simultaneously, which means that a very long time is needed for the acquisition of statistically relevant data. These technical problems have been solved by the present invention.

### SUMMARY OF THE INVENTION

The invention relates to biochips which enable the targeting of single nucleic acid molecules in predefined regions: although one end of the nucleic acid molecule is immobilized on the chip, the rest of the nucleic acid molecule remains free to fluctuate, independently of the other molecules, in solution and under conditions which allow observation by optical microscopy. The invention is based on a definition of the maximum size of the regions where the single DNA molecules bind and on the spacing necessary between two regions. By adhering to the dimensions described by the invention, it is possible to increase the density of nucleic acid molecules simultaneously observable while at the same time retaining a level of validity of the trajectories identical to that measured by conventional TPM.

The invention relates to biochips comprising a substrate, said substrate comprising at its surface isolated regions for the anchoring of a nucleic acid molecule, said isolated regions having an area of less than 1 μm², and the space between two isolated regions being at least equal to the square root of the value of said area of said isolated regions.

The invention also relates to a process for fabricating biochips according to the invention, comprising the following steps:
(a) providing a substrate, and
(b) printing on said substrate isolated regions for the anchoring of a nucleic acid molecule, said isolated regions having an area of less than 1 μm², and the space between two isolated regions being at least equal to the square root of the value of said area of said isolated regions.

The invention also relates to the use of a biochip according to the invention for studying nucleic acid molecules using the “Tethered Particle Motion” or “TPM” technique. The invention also relates to a process for studying nucleic acid molecules using the “Tethered Particle Motion” or “TPM” technique, comprising the steps of:
1) providing a biochip according to the invention,
2) treating the nucleic acid molecules so as, on the one hand, to be able to attach them to the biochip according to the invention and, on the other hand, to be able to analyze them using the “Tethered Particle Motion” or “TPM” technique,
3) studying the nucleic acid molecules using the “Tethered Particle Motion” or “TPM” technique.

The invention also relates to kits comprising:
a biochip according to the invention, and
a computer readable medium comprising instructions which can be carried out by said computer in order to implement a process for studying nucleic acid molecules using the “Tethered Particle Motion” technique according to the invention.

### DEFINITIONS

The term “biochip”, as used herein, refers to a nucleic acid chip, commonly called “DNA chip” or “RNA chip”. A biochip consists of a substrate to which nucleic acid molecules can be attached.

The term “anchoring of a nucleic acid molecule”, as used herein, refers to the attachment of a nucleic acid molecule to the substrate of the biochip.

The term “nucleic acid molecule”, as used herein, refers to a molecule of single-stranded or double-stranded DNA or of RNA.

The term “molecule for the anchoring of a nucleic acid molecule”, as used herein, refers to any molecule capable of binding, on the one hand, to the substrate and, on the other hand, to a nucleic acid molecule.

The term “isolated region for the anchoring of a nucleic acid molecule”, as used herein, refers to a “region for the anchoring of a nucleic acid molecule” which is not in contact with another “region for the anchoring of a nucleic acid molecule” of the biochip.

### DETAILED DESCRIPTION OF THE INVENTION

### Biochips

As is illustrated in FIG. 1, the invention relates to biochips 1 comprising a substrate 2, said substrate comprising at its surface isolated regions 3 for the anchoring of a nucleic acid molecule, said isolated regions having an area of less than 1 μm², and the space 4 between two isolated regions being at least equal to the square root of the value of said area of said isolated regions.

According to one particular embodiment, the biochips according to the invention are characterized in that said isolated regions have, at the surface, a layer of molecules for the anchoring of a nucleic acid molecule.

Typically, said molecules for the anchoring of a nucleic acid molecule according to the invention are anchoring proteins chosen from streptavidin, avidin, streptavidin derivatives and avidin derivatives (for the purpose of the invention, the expression “streptavidin derivatives and avidin derivatives” is intended to mean any molecule resulting from a chemical or biological modification of streptavidin or of avidin and which retains an affinity for biotin, for example neutravidin), and antibodies, for example anti-digoxigenin, anti-BSA (bovine serum albumin) or anti-carboxyfluorescein antibodies. In such an embodiment, the nucleic acid molecule is itself treated so as to bind to the anchoring molecule: one end of the nucleic acid molecule is, for example, bound to a biotin molecule (which will typically bind to a streptavidin or avidin molecule or to a derivative thereof), or to an antigen (recognized by the antibody). Said molecules for the anchoring of a nucleic acid molecule may also be oligonucleotides or functionalized oligonucleotides (amine- or thiol-functionalized). These oligonucleotides are short RNA or DNA nucleotide sequences, which are single-stranded and a few tens of bases long. The anchoring of the nucleic acid molecule will then take place by hybridization with the oligonucleotide.

According to the invention, the limitation of the size of the isolated regions of the biochip and also the definition of a minimum spacing between two isolated regions of the biochip ensures the anchoring of a single nucleic acid molecule per isolated region.

In one particular embodiment, said isolated regions of the biochips according to the invention have an area of less than or equal to approximately 0.9 μm², approximately 0.8 μm², approximately 0.7 μm², approximately 0.6 μm², approximately 0.5 μtm², approximately 0.4 μm², approximately 0.3 μm², approximately 0.2 μm², approximately 0.1 μm², approximately 0.09 μm², approximately 0.07 μm², approximately 0.05 μm², or approximately 0.04 μm².

Still in one particular embodiment, said isolated regions of the biochips according to the invention have a substantially square shape with a side of less than 1 μm (i.e. an area of less than 1 μm²), particularly less than or equal to approximately 900 nm (i.e. an area of at most approximately 0.81 μm²), more particularly less than or equal to approximately 800 nm (i.e. an area of at most approximately 0.64 μm²), more particularly less than or equal to approximately 700 nm (i.e. an area of at most approximately 0.49 μm²), more particularly still less than or equal to approximately 600 nm (i.e. an area of at most approximately 0.36 μm²), even more particularly less than or equal to approximately 500 nm (i.e. an area of at most approximately 0.25 μm²), approximately 400 nm (i.e. an area of at most approximately 0.16 μm²), approximately 300 nm (i.e. an area of at most approximately 0.09 μm²) or even approximately 200 nm (i.e. an area of at most approximately 0.04 μm²). According to the invention, the expression “side less than or equal to . . . ” is intended to mean that the side of the square has a length “less than or equal to . . . ”.

According to another particular embodiment, said isolated regions of the biochips according to the invention have a substantially round shape having an area of less than 1 μm², particularly less than or equal to approximately 0.9 μm², approximately 0.8 μm², approximately 0.7 μm², approximately 0.6 μm², approximately 0.5 μm², approximately 0.4 μm², approximately 0.3 μm², approximately 0.2 μm², approximately 0.1 μm², approximately 0.09 μm², approximately 0.07 μm², approximately 0.05 μm², or approximately 0.04 μm².

According to the invention, the space 4 between two isolated regions of the biochip is at least equal to the square root of the value of said area of said isolated regions. For example, if the area of said isolated regions is 0.5 μm², then the space between these two isolated regions will be at least equal to the square root of 0.5 μm², i.e. at least equal to approximately 700 nm. When two isolated regions do not have an identical area, the space between these two isolated regions then corresponds to the square root of the average of the two areas: if a first region has an area of 1 μm² and the second an area of 0.5 μm², the average of the two areas will be approximately 0.75 μm², and the space between these two regions will then be approximately 860 nm.

Typically, the substrate of the biochips according to the invention is chosen from an inorganic substrate; an organic substrate, in particular a polymer substrate; and a metal substrate.

In one particular embodiment, the substrate is a substrate functionalized with epoxides, i.e. a substrate of which at least one of the surfaces is covered with a layer of molecules which have epoxide chemical functions capable of binding the free amines present on the anchoring proteins. Typically, an “epoxidized” substrate according to the invention is a substrate covered with a self assembled monolayer or SAM of silanes carrying an epoxide function at their end. The silanes are typically chosen from: silane (SiₙH₂ₙ₊₂; n representing a number from 1 to 15), silicone alkoxide, polysilane, silanol, tetraalkoxysilane, trimethylsilane, vinyltrichlorosilane, trichlorosilane, dimethyldichlorosilane, methyldichlorosilane, diethyldichlorosilane, allyltrichlorosilane (stabilized), dichlorosilane, ethyl silicane, dimethyldichlorosilane, silicoheptane, trimethylsilyl azide, trimethylchlorosilane, 3-mercaptopropyltrimethoxysilane, methyltrimethoxysilane, methyl silicane, tetraethylorthosilane, tetramethoxysilane, silane coupling agent, silicobromoform, silicoiodoform, phenyltrimethoxysilane, alkylsilanediol, chloromethylphenyltrimethoxysilane, hydroxyorganosilane, polyalkoxysilane, cyclopentasilane, and dimethyldichlorosilane.

In one particular embodiment, the substrate is a glass coverslip functionalized with epoxide functions.

According to one embodiment, the binding of the anchoring proteins to the “epoxidized” substrate is carried out by the technique described by Rusmini et al. in the review “Protein immobilization strategies for protein biochips” in the journal Biomacromolecules, 2007.

In another particular embodiment, the substrate is a substrate carrying succinimidyl ester or isothiocyanate end groups, or else the substrate is silanized with amino or thiolated or epoxidized silanes and then bonded to appropriate PEG/PEG-biotin molecules (PEG: polyethylene glycol).

In one particular embodiment, the substrate is a glass coverslip functionalized with a PEG/PEG-biotin mixture.

According to the invention, each isolated region of the biochip enables the anchoring of a single nucleic acid molecule (i.e. the attachment of one nucleic acid molecule per region), provided that the characteristic dimension of said nucleic acid molecule is greater than half the square root of the value of the area of the isolated region to which said nucleic acid molecule (NA) is attached. The expression “characteristic dimension of a nucleic acid molecule” (or “D_{char}”) is intended to mean either the characteristic dimension of a nucleic acid molecule not coupled to a bead, or the characteristic dimension of a nucleic acid molecule coupled to a bead. The characteristic dimension D_{char} of the NA or of the NA+ bead couple is calculated as follows:
for a nucleic acid molecule (NA) not coupled to a bead:

D_{char}=2R_{NA}

where R_{NA} corresponds to the end-to-end length of the nucleic acid molecule, equivalent to the Flory radius, with R_{NA}=2Lₚ (L/2Lₚ)^{3/5} where L is the length of the nucleic acid molecule studied (determined by multiplying the number of bases or of base pairs of the NA molecule by the average distance between bases or between base pairs, which is approximately 0.34 nm) and Lₚ is the NA persistence length. The NA persistence length Lₚ corresponds approximately to the length of 150 base pairs for a double-stranded nucleic acid (i.e. approximately 51 nm) and approximately to the length of 3 bases for a single-stranded nucleic acid (i.e. approximately 1.02 nm);
for a nucleic acid molecule (NA) coupled to a bead:

D_{char}=D_{bead}+R_{NA}

where R_{NA} is as defined previously and D_{bead} corresponds to the diameter of the bead. For example, for DNA molecules of 300 by attached to a bead which is 300 nm in diameter, the distance between base pairs being 0.34 nm, R_{NA}=102 nm, and D_{char}=300+102=402 nm. Thus, in order to ensure the attachment of a single molecule of this DNA/bead couple per isolated region, the area of the isolated region will have to be less than 0.646 μm² (i.e. a square with a side of at most 804 nm).

In the case of the TPM application, said isolated regions of the biochips according to the invention typically enable the anchoring of a nucleic acid molecule comprising 300 to 3000 base pairs. If the nucleic acid is single-stranded, said isolated regions of the biochips according to the invention typically enable the anchoring of a nucleic acid molecule comprising 4500 to 45000 nucleotides.

According to one embodiment, the biochips also comprise an observation chamber for both the introduction of various solutions at the level of the isolated regions, and also the observation of the chip, in particular under an optical microscope. According to this embodiment, the chip comprises a coverslip positioned above the substrate, said coverslip comprising at least two openings for the introduction of solutions at the level of the isolated regions, the whole assembly defining an observation chamber. Typically, the coverslip is a glass, poly(methyl methacrylate) or polycarbonate coverslip, approximately 0.5 mm to 1 mm thick, bonded or placed on the substrate. The coverslip may be typically bonded on the substrate using double-sided adhesive, in particular SecureSeal® (Grace Bio-Labs, 0.12 mm or 0.24 mm thick).

### Process for fabricating biochips

The invention also relates to a process for fabricating biochips according to the invention, comprising the following steps:
(a) providing a substrate, and
(b) printing on said substrate isolated regions for the anchoring of a nucleic acid molecule, said isolated regions having an area of less than 1 μm², and the space between two isolated regions being at least equal to the square root of the value of said area of said isolated regions.

According to one embodiment, the substrate is cleaned and treated before undertaking step (b).

Typically, the cleaning of the substrate can be carried out by sonication of the substrate in an ultrasound bath in ethanol for 5 min, followed by oxygen plasma treatment (typically approximately 15 min, at a power of 80 W with 0.1 mbar O₂). The cleaning can also be carried out with a sulfochromic mixture (approximately 1 h in a solution of H₂SO₄ typically containing 70 g/l K₂/Na₂Cr₂O₇ and 50 ml/l H₂O) or else with a “Piranha” mixture (1 h in a solution of 7/3 v/v H₂SO₄ and H₂O₂), the latter two treatments being followed by thorough rinsing with deionized water.

According to this embodiment, the substrate, once cleaned, is treated so as to attach said molecules for the anchoring of a nucleic acid molecule. When the anchoring molecules are anchoring proteins, this treatment typically consists of a silanization of the substrate, for example by immersion of the substrate for 1h30 in a solution of isopropanol containing 2.5% of 3-glycidoxypropyldimethoxymethylsilane (GPDS), 0.05% of benzyldimethylamine and 0.5% of deionized water. The substrate is then thoroughly rinsed, typically with deionized water, then dried (for example, under a stream of an inert gas, for example nitrogen, and then in an oven at 110° C. for 15 minutes). This step confers on the substrate epoxide functions capable of reacting with free amine functions of the anchoring proteins. It is thus possible to attach anchoring proteins to the substrate (the anchoring protein reacting, on the one hand, with the epoxide functions of the substrate and, on the other hand, with the nucleic acid molecule, previously functionalized and coupled to a bead). The substrate thus cleaned and treated can generally be stored for up to two weeks under vacuum and in the dark before step (b).

According to one embodiment of the invention, step (b) consists in printing on the substrate, in said isolated regions, a layer of molecules for the anchoring of a nucleic acid molecule.

The printing on the substrate, in said isolated regions, of a layer of molecules for the anchoring of a nucleic acid molecule is typically carried out by the molecular stamping or “microcontact printing” method (described in particular in WO 96/29629), cf. FIG. 3. This soft lithography technique consists in bringing the substrate into contact with an elastomeric stamp structured in the form of micrometer-sized patterns covered with anchoring molecules. This method enables the formation, on the surface thereof, of isolated regions with a layer of anchoring molecules.

A silicon wafer (wafer of semi-conducting material) which has a network of square patterns of submicrometric size is typically used as a mold for the fabrication of the microstructured elastomeric stamps. The patterns are spaced out by a few μm (for example 2.5 μm), so as to avoid adjacent “nucleic acid molecule/bead” couples influencing one another, and typically etched to a depth of 1 μm.

The maximum dimension of the patterns of the wafer is calculated such that the isolated regions that will be “printed” on the substrate of the biochip only allow the anchoring of a single nucleic acid molecule (NA) or of an NA-bead couple. The patterns of the wafer thus have an area (corresponding, after printing on the substrate, to the area of said isolated regions) which is directly dependent on the characteristic dimension D_{char} of the NA or of the NA+bead couple that it is desired to attach to the biochip, as is previously explained. The elastomer stamp can then be typically obtained by crosslinking, at 60° C. for 48 h, of polydimethylsiloxane (for example PDMS Sylgard 184, Dow Corning) deposited on the microstructured silicon wafer. The stamp bears the inverse topographic patterns of those present on the silicon wafer (Xia et al., 1998) the sizes of which define those of the patterns of anchoring molecules that will be deposited on the substrate. The microstructured face of the PDMS stamp is subsequently typically brought into contact with a buffered solution (for example, phosphate buffered saline, 150 mM NaCl, pH7.4) of anchoring molecules for 30 seconds. Neutravidin or an anti-digoxigenin antibody at a concentration of 10 μg/ml is typically used as anchoring molecules for specifically binding a biotinylated or digoxigenin-functionalized DNA. The microstructured face is then rinsed, for example with deionized water, and dried, for example under a stream of an inert gas (for example, nitrogen). The microstructured face, inked in this way, is then typically affixed for approximately 10 s on the substrate. In the absence of applied external force, a conformal contact (i.e. a total contact between the two surfaces) is typically established between the substrate and the patterns of the PDMS stamp. It results, under the conditions previously defined, in the transfer of a monolayer of anchoring molecules from the patterns of the PDMS stamp to the substrate. The stamp, removed for example after 10 s of contact, can be cleaned (sonication for 5 min in an equivolume ethanol/water mixture) so as to be subsequently reused.

The printing can also be carried out by the “lift-off” method (von Philipsborn et al., Nat Protoc. 2006; 1322-8; and WO2010/020893).

Typically, according to this method, a monolayer of anchoring proteins is adsorbed onto the planar face of a PDMS stamp (the face opposite the bottom of the dish in which the PDMS is crosslinked). Bringing it into conformal contact for 1 min with the surface, activated with an oxygen plasma (0.04 mbar O₂, 1 minute at 200 W), of a microstructured silicon wafer leads to the transfer of the proteins onto the silicon. After separation, only the proteins located opposite the patterns of the silicon mould remain on the planar surface of the PDMS stamp, which is immediately applied against the epoxide-functionalized glass coverslip for 10 s (see FIG. 5). This method requires rigorous cleaning of the silicon wafer for repeated use thereof.

The printing can also be carried out by the “inverted print” method (Cherniayskaya et al., 2002).

This method requires the use of a silicon wafer which has patterns which are inverted compared with those of the two methods previously described (see FIG. 7), and typically 120 nm deep. The stamp of PDMS crosslinked on the wafer is then typically deposited on a drop, formed on a hydrophobic surface (for example of Parafilm), of a solution of anchoring proteins for a few minutes. After rinsing, for example with deionized water, and drying under a stream of inert gas (for example, nitrogen), the stamp is brought into contact (3s) repeatedly with a freshly cleaved mica surface or a hydrophobic surface or surface made hydrophobic, before being finally affixed on a substrate (typically previously epoxidized) by applying a considerable pressure, typically for 30 s. The repeated bringing of the stamp into contact with the mica surface or the hydrophobic surface or surface made hydrophobic will remove the proteins of the surface of the patterns of the PDMS stamp. The lateral and vertical dimensions of the microstructures of the PDMS stamp make it possible, during the application of a pressure, to transfer anchoring molecules located in the inverted (hollow) patterns. This variant of molecular stamping makes it possible to deposit patterns of a size between 1 μm and 200 nm.

Application of the biochips according to the invention to the “TPM” technique The invention also relates to the use of a biochip according to the invention for studying nucleic acid molecules using the “Tethered Particle Motion” or “TPM” technique. In one particular embodiment, the characteristic dimension of said nucleic acid molecules is greater than half the square root of the value of the area of said isolated regions of the biochip.

The invention also relates to a process for studying nucleic acid molecules using the “Tethered Particle Motion” or “TPM” technique, comprising the steps of:
1) providing a biochip according to the invention,
2) treating the nucleic acid molecules so as, on the one hand, to be able to attach them to the biochip and, on the other hand, to be able to analyze them using the “Tethered Particle Motion” or “TPM” technique,
3) studying the nucleic acid molecules using the “Tethered Particle Motion” or “TPM” technique.

In one particular embodiment, said process for studying nucleic acid molecules using the “Tethered Particle Motion” or “TPM” technique is characterized in that the characteristic dimension of said nucleic acid molecules is greater than half the square root of the value of the area of said isolated regions of the biochip.

The biochips according to the invention enable the high-throughput acquisition, using the “Tethered Particle Motion” or “TPM” technique, of measurements on single (one molecule per isolated region) nucleic acid molecules (double-stranded or single-stranded, DNA or RNA) and the real-time analysis thereof through the simultaneous observation of a set of molecules immobilized on the sites of a network. TPM consists in observing, by optical microscopy, the Brownian motion of a bead bonded to the free end of a single DNA molecule immobilized on a glass coverslip by the other end (FIG. 2). The amplitude of the Brownian motion of the bead depends on the length of the DNA molecule. Any of the conformational changes in DNA that are induced by external factors (proteins, ions, temperature) which induce a change in the apparent length of the DNA molecule can be analyzed by TPM, which leads to a very large number of applications. Examples of applications are given hereinafter:
measurement of the actual length of a double-stranded DNA, in particular described in Schafer et al., Nature. 1991; 352(6334): 444-8, and more recently in Nelson et al, “Tethered Particle Motion as a Diagnostic of DNA Tether Length”, J. Phys. Chem B, 2006, 110, 17260,
characterization of a conformational change in DNA as a function of time, in particular described in Finzi and Gelles, “Measurement of lactose repressor loop formation and breakdown in single DNA molecules”, Science 1995, 267(5196): 378, and more recently in Laurens et al., “Dissecting protein-induced DNA looping dynamics in real time” Nucleic Acids Res. 2009; 37(16): 5454-64,
measurement of the actual length of a double-stranded DNA by dark field TPM with gold colloids, described in Brinkers et al, “The persistence length of double stranded DNA determined using dark field tethered particle motion”, J Chem Phys, 2009, 130, 215105.

Step (2) typically consists in functionalizing the nucleic acid molecules in a distinct manner at their two ends, so as to specifically bond, on the one hand, a bead and, on the other hand, the anchoring molecules deposited on the substrate. This step is well known to those skilled in the art specializing in the TPM technique.

The double-stranded nucleic acid molecules are typically obtained by PCR in the presence of primers tagged either with a biotin or with a digoxigenin (see FIG. 9). The single-stranded nucleic acid molecules are, for their part, typically functionalized in a distinct manner at their 5′ and 3′ ends, as in particular described in Lambert et al., Biophys. J. 2005 (90), 3672.

Examples of beads which are suitable for the invention are latex or polymer particles from 5 to 800 nm in diameter, which may be fluorescent or nonfluorescent (typically Fluospheres® or Qdot® nanocrystals from Invitrogen), and which are typically covered with anti-digoxigenin antibodies (which bind to a digoxigenin unit present at one end of a nucleic acid molecule), with streptavidin, with avidin, or with a derivative of streptavidin and of avidin (which binds to a biotin unit present at one end of a nucleic acid molecule) or else which have carboxylic acid functions at their surface (enabling covalent bonding with an amine-tagged nucleic acid molecule).

Other examples of beads that are suitable for the invention are gold colloids (typically those from British Biocell International) from 10 to 200 nm in diameter. The gold colloids can bond directly to a nucleic acid molecule functionalized with a thiol function. The gold colloids may also be covered, for example, with anti-digoxigenin antibodies (which bind to a digoxigenin unit present at one end of a nucleic acid molecule) or with streptavidin, with avidin, or with a derivative of streptavidin and of avidin (which binds to a biotin unit present at one end of a nucleic acid molecule).

Equimolar solutions of nucleic acid molecules (NAs) (for example in a PBS buffer, pH 7.4, 0.1 mg/ml BAS, 1 mg/ml pluronic F-127) and of beads (for example in a PBS buffer pH 7.4, 0.1 mg/ml BSA, 0.1% Triton, 0.05% Tween 20, 1 mg/ml pluronic F-127) are typically mixed at ambient temperature for 1 h. Under these conditions, a mixture comprising beads not bonded to an NA molecule (approximately 37%), beads bonded to 1 NA (approximately 37%) and beads bonded to several NAs (approximately 26%) is typically expected. The separation of the beads with or without nucleic acid molecule can, for example, be carried out using functionalized magnetic beads which bind the “nucleic acid molecule/bead” complexes and not the beads alone.

The sample containing the “nucleic acid molecule/bead” complexes (at a concentration typically between 20 and 100 pM) is then injected onto the biochip (typically in the biochip observation chamber). The minimum incubation time is 3 h, and then rinsing is carried out.

The biochip is then typically placed on the platform of an optical microscope. Depending on the nature of the bead (fluorescent or nonfluorescent particle, or gold colloid), the microscope is used in epifluorescence mode (for the fluorescent particles) or light or dark field mode (for the nonfluorescent particles and the gold colloids). The presence of the beads attached in an ordered manner on the patterns of anchoring proteins makes it possible to rapidly bring the region of interest into focus. The motion of the beads is then typically studied using a computer program (software) which integrates the calculations for determining the dynamic parameters of the beads (the amplitude of the motion of the bead, its anchoring point, the motion asymmetry factor), as in particular described in the experimental section of the invention.

### Kits

The invention also relates to kits comprising:
a biochip according to the invention, and
a computer-readable medium comprising computer-executable instructions for implementing a process for studying nucleic acid molecules using the “Tethered Particle Motion” technique according to the invention.

The invention is also described by means of the figures and examples hereinafter, given only by way of illustration.

### BRIEF DESCRIPTION OF THE FIGURES

FIG. 1: Diagram of a biochip according to the invention. Biochip 1 comprising a substrate 2, said substrate comprising at its surface isolated regions 3 for the anchoring of a nucleic acid molecule, said isolated regions having an area of less than 1 μm², and the space 4 between two isolated regions being at least equal to the square root of the value of said area of said isolated regions.

FIG. 2: Example of a diagram of the principle of TPM. A DNA molecule (d) comprising at each of its ends a biotin molecule (c) and a digoxigenin molecule (e), respectively, is bound, on the one hand, to a neutravidin unit (b) itself attached to a substrate (a), and via the other end to a bead covered with anti-digoxigenin antibodies (g). The Brownian motion (f) of the bead can then be studied using the TPM method.

FIG. 3: Conventional “microcontact printing” method. (A) crosslinking of a PDMS stamp; (B) inking of the stamp; (C) rinsing and drying; (D) molecular stamping; (E) obtaining a functionalized surface. (6) PDMS stamp; (7) microstructured silicon wafer; (8) solution of proteins or proteins after drying; (9) glass coverslip.

FIG. 4: Image of a network of patterns of anchoring proteins (neutravidin labeled with TRITC, TetramethylRhodamine IsoThioCyanate) deposited by conventional microcontact printing on an epoxide-functionalized glass coverslip. A zone of nonstructured deposition, due to the collapse of the stamp on the glass coverslip, is visible on the left part of the image. The square patterns have sides of 600 nm and are 2.5 μm apart.

FIG. 5: Subtractive “Microcontact Printing” method—Variant 1. (A) inking of the flat stamp; (B) rinsing and drying; (C) subtraction of a part of the layer of proteins of the stamp; (D) molecular stamping; (E) obtaining a functionalized surface. (10) solution of proteins or proteins after drying; (11) flat PDMS stamp; (12) microstructured silicon wafer; (13) glass coverslip.

FIG. 6: image of a network of patterns of anchoring proteins (neutravidin labeled with TRITC) deposited via variant 1 of the microcontact printing method on an epoxide-functionalized glass coverslip. The square patterns have sides of 800 nm and are 3 μm apart.

FIG. 7: Inverted subtractive “Microcontact Printing” method-variant 2. (A) crosslinking of a PDMS stamp; (B) inking of the stamp; (C) rinsing and drying; (D) removal of the surface proteins (step repeated several times, typically four times); (E) molecular stamping with external pressure; (F) obtaining a functionalized surface. (14) PDMS stamp; (15) microstructured silicon wafer; (16) hydrophobic surface; (17) solution of proteins or proteins after drying; (18) mica surface; (19) glass coverslip.

FIG. 8: image of a network of patterns of anchoring proteins (neutravidin labeled with TRITC) deposited via variant 2 of the microcontact printing method on an epoxide-functionalized glass coverslip. The square patterns have sides of 400 nm and are 5 μm apart.

FIG. 9: Diagram of functionalization of a DNA molecule for bonding thereof to a bead and to a protein pattern deposited on a substrate. (20) neutravidin pattern deposited on a substrate; (21) biotin; (22) nucleic acid molecule; (23) digoxigenin; (24) anti-digoxigenin bead; (25) anti-digoxigenin pattern deposited on a substrate; (26) digoxigenin; (27) nucleic acid molecule; (28) biotin; (29) neutravidin bead.

FIG. 10: A) image of a network of 5 μm-sided patterns, spaced 10 μm apart, of anchoring proteins (neutravidin labeled with TRITC) onto which bead/DNA complexes (0.2 μm, yellow-green fluorescent (505/515) NeutrAvidin® labeled Fluospheres® microspheres from Molecular Probes, Invitrogen Detection Technologies) are specifically targeted. B) image of a network of patterns of anchoring proteins (neutravidin labeled with TRITC) deposited by conventional microcontact printing on an epoxide-functionalized glass coverslip. The square patterns have sides of 600 nm and are spaced 2.5 μm apart. C) image of the bead/DNA complexes (FITC fluorescent beads) specifically targeted on the network of the patterns of anchoring proteins.

FIG. 11: Amplitude of the motion of beads 300 nm in diameter (in nm) as a function of the length of the bonded DNA bonding it to the substrate (in base pairs, bp). (▪)TPM measurements on structured network; (♦) conventional TPM measurements.

FIG. 12: Amplitude of the motion of a bead 300 nm in diameter bonded to a DNA molecule (in bp) degraded by T7 exonuclease over time (in seconds).

FIG. 13: Histogram (in number of beads followed) of the rates of degradation by the T7 exonuclease (in nucleotides per second).

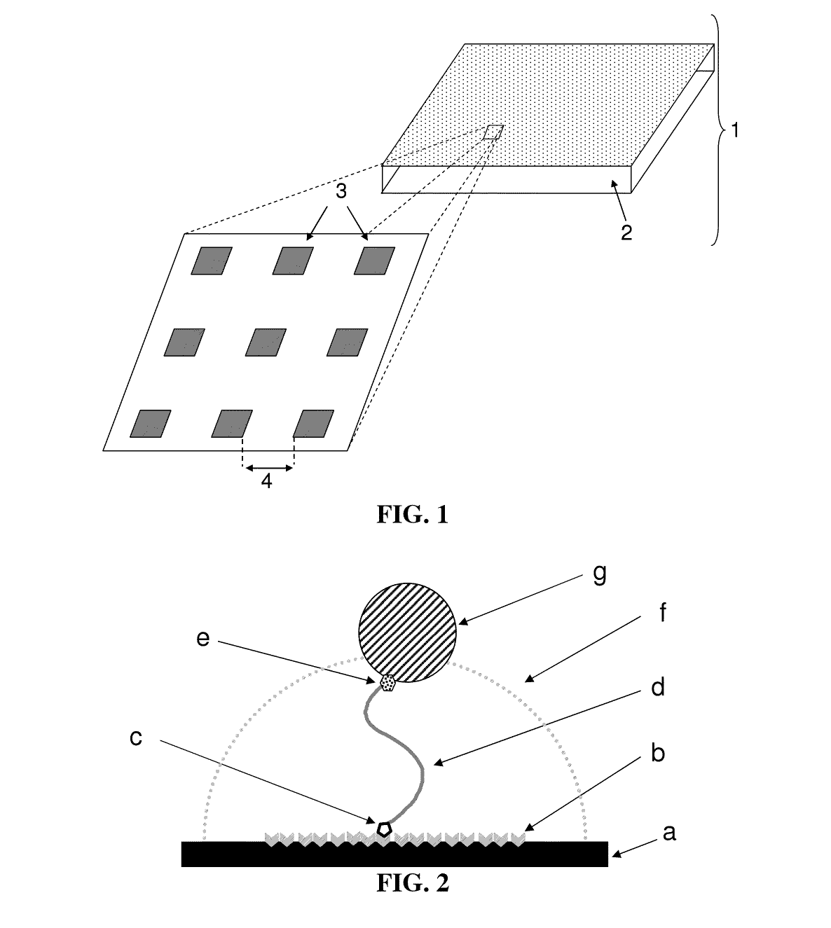

### EXAMPLES

We fabricated biochips according to the invention by following the steps described hereinafter.

### 1. Structured Functionalization of a Glass Coverslip

This step was carried out by “microcontact printing”. In order to ensure the attachment of a single object per site, we demonstrated that the size of the sites must be of the same order as or less than that of the object. In our tests, the substrate of the biochip is a glass coverslip (or slide) with dimensions of 24×18 mm². Moreover, the nucleic acids tested are DNA molecules that we previously coupled to beads, and the size of the protein patterns printed on the glass coverslip was of the order of that of the DNA/bead complex.

### 1.1 Silanization of the Glass Coverslip

In order to bond to the glass coverslip anchoring proteins that will act as active sites for the binding of DNA molecules, the coverslip was covered with a self-assembled monolayer of silanes. Prior cleaning of the slides was carried out by sonication in an ultrasound bath in ethanol for 5 min, followed by a treatment with a sulfochromic mixture (approximately 1 h in a solution of H₂SO₄ containing 70 g/l K₂/Na₂Cr₂O₇ and 50 ml/l H₂O) followed by thorough rinsing with deionized water.

Next, we carried out a silanization protocol consisting of the immersion of the cleaned glass slides as described above for 1 h30 in a solution of isopropanol containing 2.5% of 3-glycidoxypropyldimethoxymethylsilane (GPDS), 0.05% of benzyldimethylamine and 0.5% of deionized water. After thorough rinsing with deionized water and drying (under a stream of an inert gas (for example, nitrogen) then in an oven at 110° C. for 15 minutes), we stored the coverslips for up to two weeks under vacuum and in the dark.

### 1.2 Fabrication of the Microstructured Elastomeric Stamp

We used a silicon wafer (wafer of semi-conducting material) having a network of square patterns of submicrometric size as a mould for fabricating microstructured elastomeric stamps. The patterns were spaced a few pm apart (typically 2.5 μm in order to avoid adjacent DNA/bead couples influencing one another) and etched to a depth of 1 μm. The maximum dimension dₘₐₓ of their side, ensuring the binding of a single DNA/bead couple per anchoring protein pattern, is defined as a function of the Flory radius of the molecules R_{DNA} and of the diameter D_{bead} of the beads according to the relationship:
dₘₐₓ≦2(D_{bead}+R_{DNA}) where R_{DNA}=2Lₚ(L/2Lₚ)^{3/5} with L being the length of the molecule studied and Lₚ the persistence length of the DNA.
We used beads 300 nm in diameter.

For DNA molecules of 798 bp, R_{DNA}=183 nm and dₘₐₓ966 nm. For DNA molecules of 2080 bp, R_{DNA}=326 nm and dₘₐₓ≦1252 nm.
We tested DNA molecules corresponding to an amplification of fragments 1063-1861bp and 4625-1861bp of the pAPT72 plasmid (798 by and 2080 bp) (the pAPT72 plasmid is described by Polard et al. in EMBO J., vol.11, no.13, pp.5079-5090, 1992).

The patterns of the silicon wafer are 1 μm-sided, 0.8 μm-sided or 0.6 μm-sided squares (the wafer has three regions with square patterns of different dimensions). We obtained an elastomeric stamp by crosslinking, at 60° C. for 48 h, polydimethylsiloxane (PDMS Sylgard 184, Dow Corning) deposited on the silicon wafer. The stamp bears the inverse topographic patterns of those present on the silicon wafer, the sizes of which define those of the protein patterns which are deposited on the glass coverslip.

### 1.3 Molecular Stamping

We subsequently brought the microstructured face of the PDMS stamp into contact with a buffered solution (phosphate buffered saline, 150 mM NaCl, pH7.4) of anchoring proteins for 30 seconds. The anchoring protein used was neutravidin at a concentration of 10 μg/ml, which makes it possible to specifically bind a biotinylated DNA.

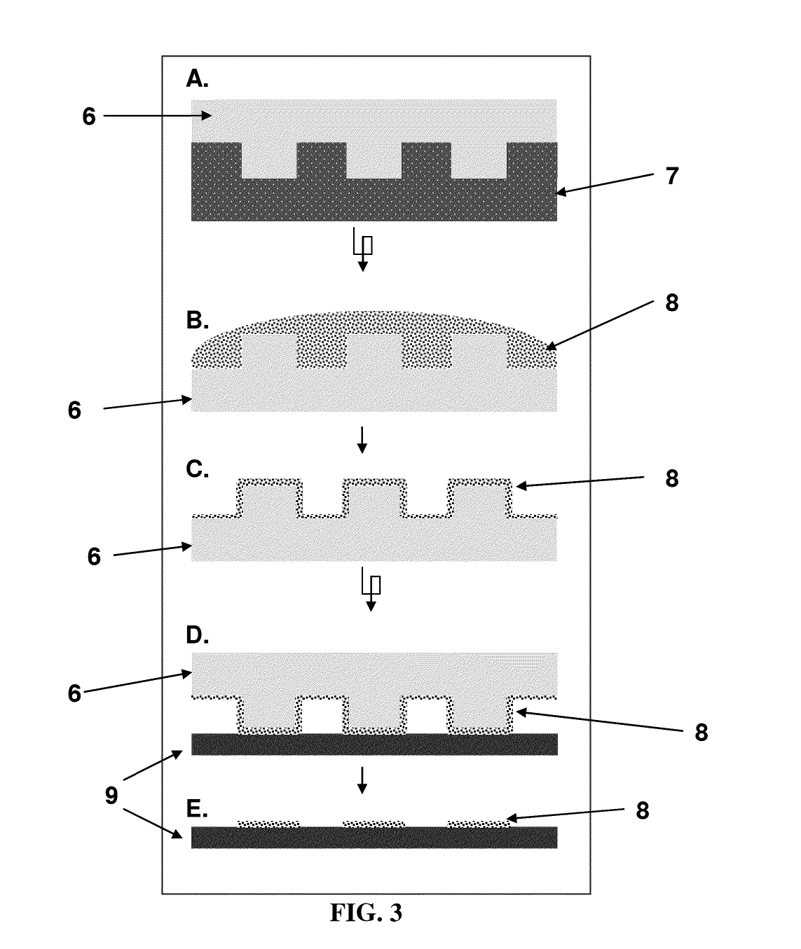

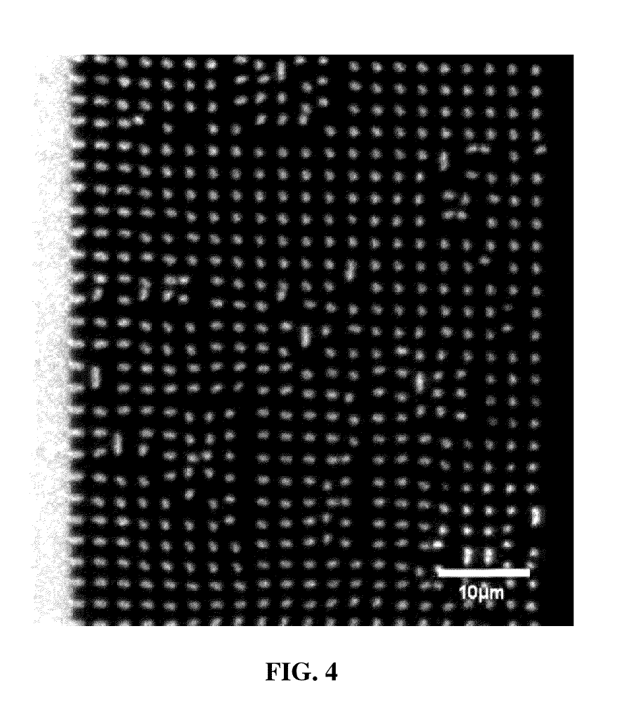

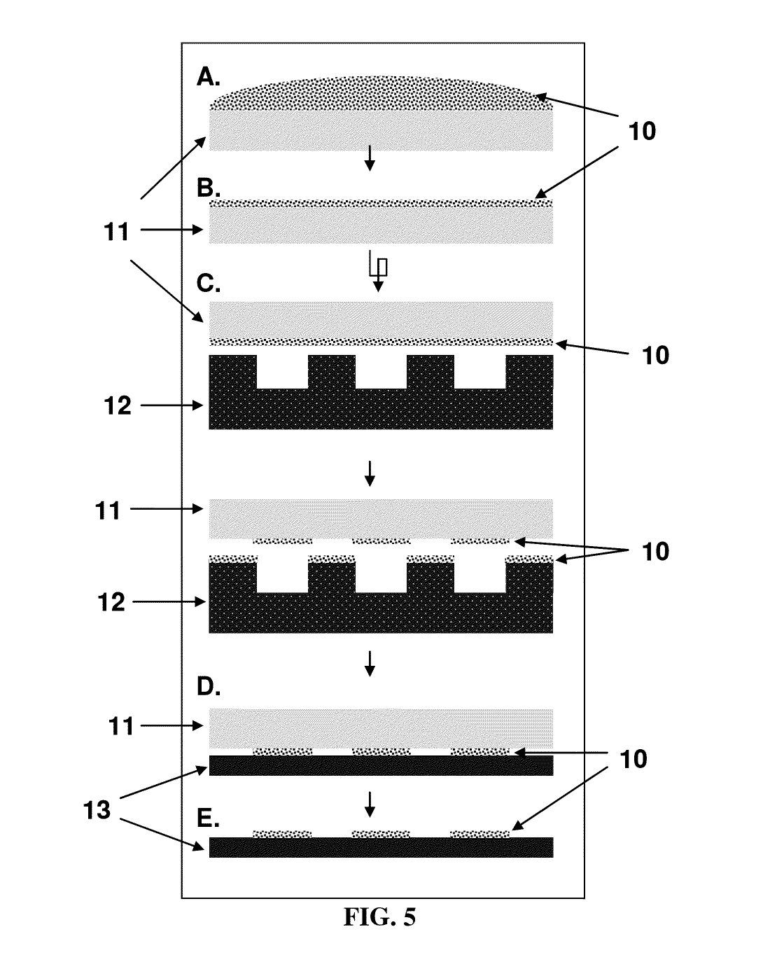

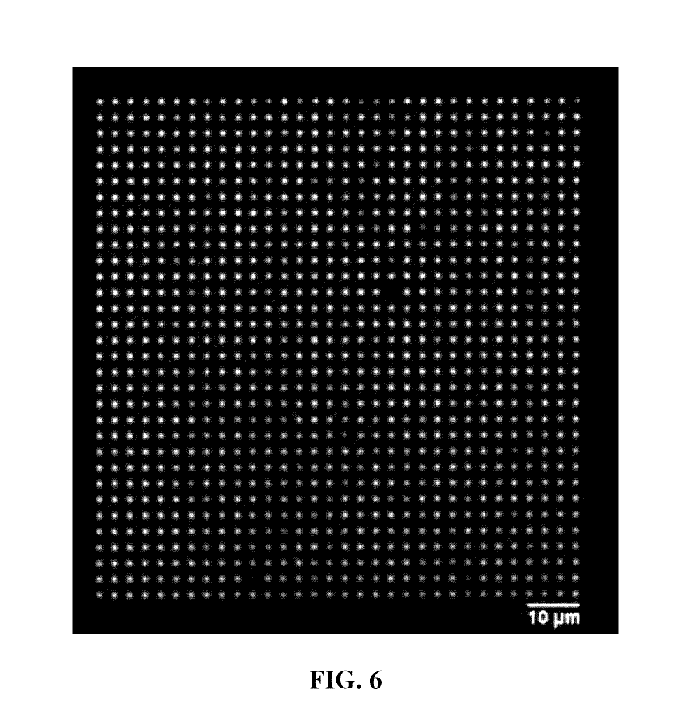

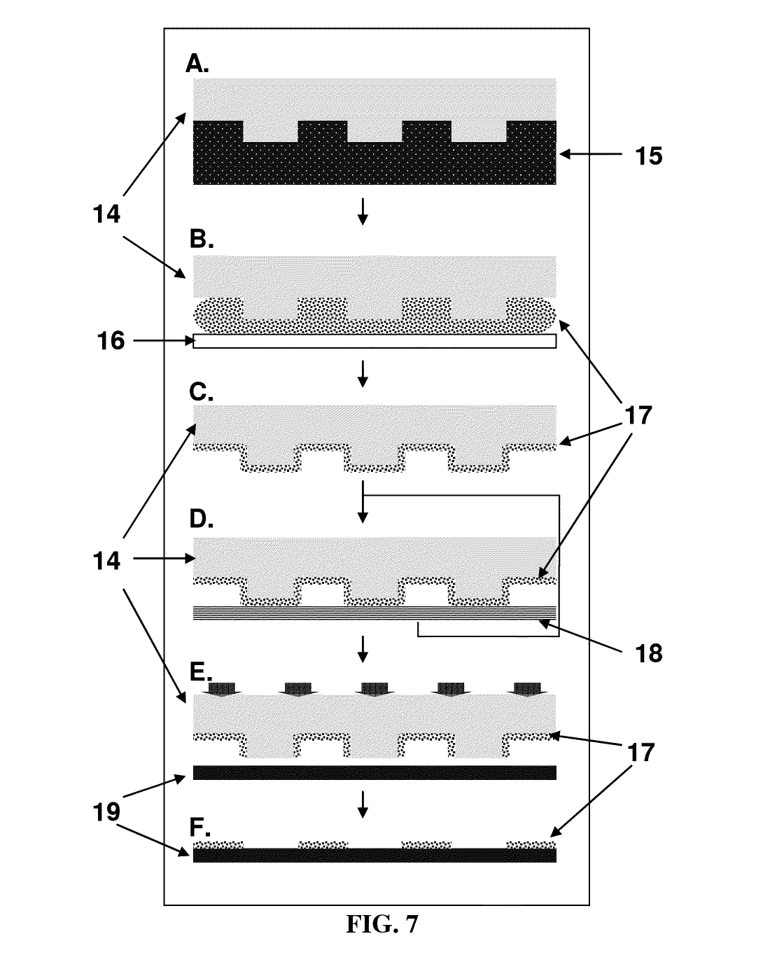

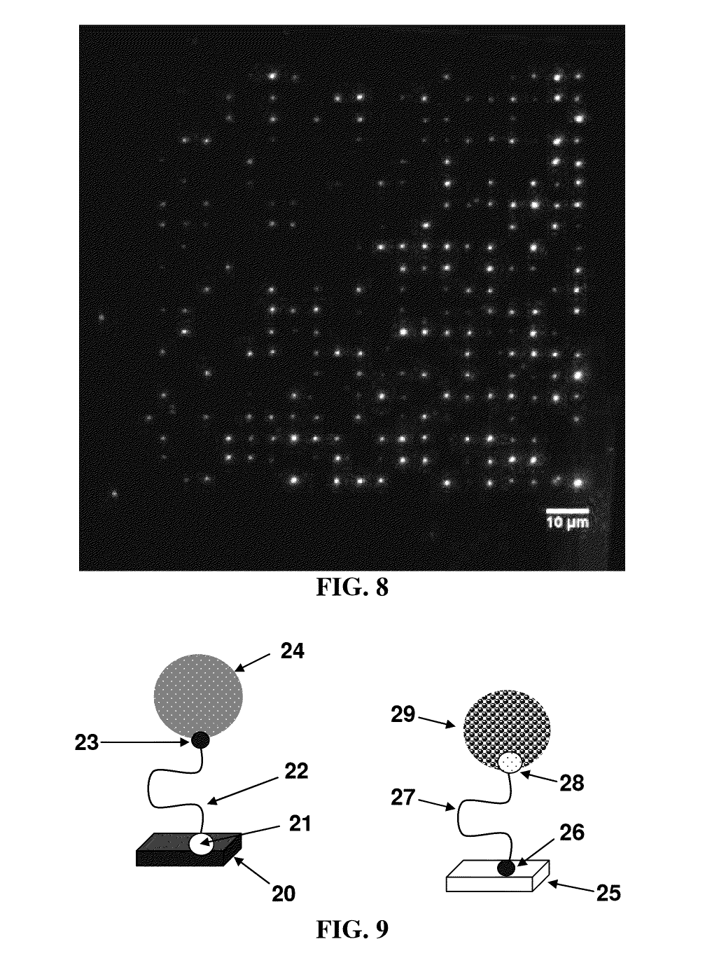

We subsequently rinsed the microstructured face with deionized water and then dried it under a stream of inert gas (for example, nitrogen). The microstructured face inked in this way was subsequently manually affixed on the epoxide-functionalized glass coverslip for 10 s (cf. point 1.1). In the absence of applied external force, a conformal contact, made possible by the elastic properties of the stamp and the relative smoothness of the glass, was established between the epoxide-functionalized glass coverslip and the patterns of the PDMS stamp. It resulted, under the conditions previously defined, in the transfer of a monolayer of proteins from the patterns of the PDMS stamp to the glass coverslip (see FIG. 3). The stamp was removed after 10 s of contact and was then cleaned by sonication for 5 min in an equivolume ethanol/deionized water mixture for subsequent reuse thereof.

2. Preparation of the observation chamber We subsequently cut up a sheet of double-sided adhesive (for example, SecureSeal™ (Grace Bio-Labs, 0.12 mm thick)) and we then stuck it to the epoxide-functionalized glass coverslip so as to form an observation chamber of reduced dimensions (typically cross section equal to 20×4 mm²) around the microstructured deposit of proteins. A poly(methyl methacrylate) strip (4 mm thick), pierced with two holes (facing the chamber) allowing the introduction of various solutions into the chamber either by direct injection using a pipette, or by perfusion (syringe driver or peristaltic pump system), was affixed on the coverslip in order to constitute the upper face of the chamber.

The chamber containing the anchoring protein patterns deposited on the glass coverslip was then, firstly, rinsed (10×chamber volume) with a passivation solution containing BSA (0.1 mg/ml), polyethylene glycol-propylene glycol (Pluronic® F-127, 1 mg/ml) and very highly negatively charged molecules (for example, heparin ˜12 kD, 0.15 mg/ml) in a PBS buffer, pH 7.4. This step made it possible, on the one hand, to remove the anchoring proteins not bound to the glass coverslip, and on the other hand, to protect the surface of the glass coverslip outside the patterns against the nonspecific adsorption of the bead-DNA complexes.

3. Preparation of the Samples to be Analyzed and Introduction into the Observation Chamber

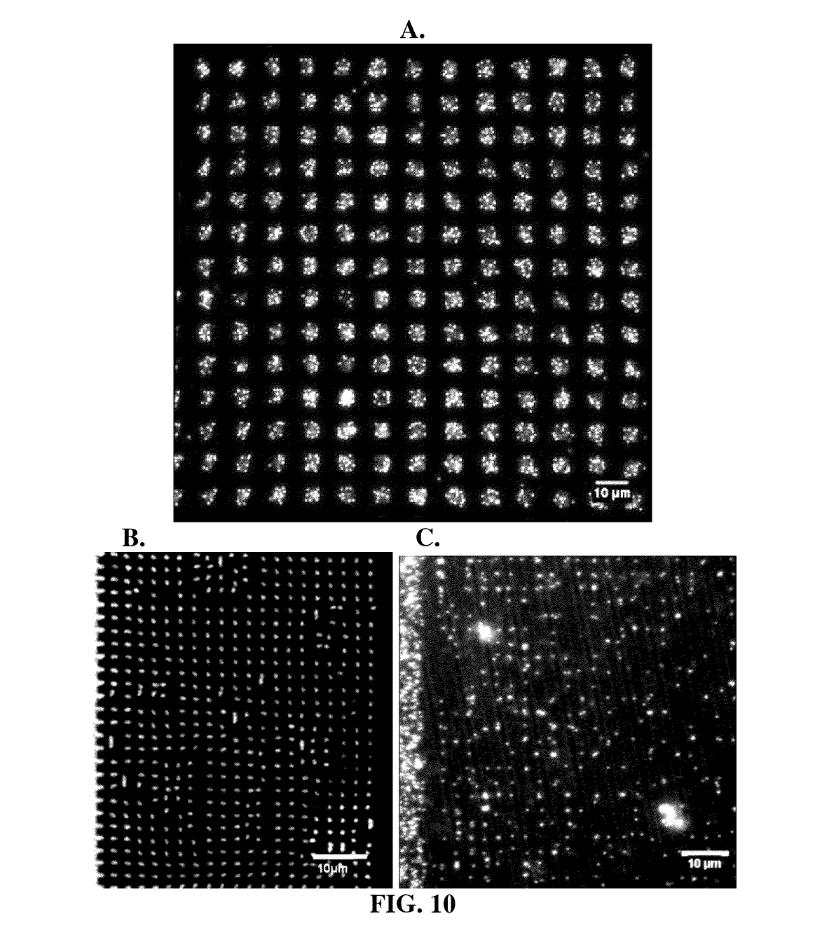

DNA molecules were functionalized in a distinct manner at their two ends so as to specifically bind, on the one hand, a bead and, on the other hand, the anchoring proteins deposited on the glass coverslip. The double-stranded DNA molecules were obtained by PCR in the presence of primers functionalized with a biotin or a digoxigenin at their 5′ end (see FIG. 9). The test experiments were carried out with double-stranded DNA molecules of a size between 401 and 2080 bp.

The beads used were fluorescent particles 300 nm in diameter, covered with anti-digoxigenin antibodies (“Anti Digoxigenin fluorescent particles”, Indicia Biotechnology®).

Equimolar solutions of DNA (in a PBS buffer, pH 7.4, 0.1 mg/ml BSA, 1 mg/ml Pluronic F-127) and of beads (PBS buffer, pH 7.4, 0.1 mg/ml BSA, 0.1% Triton® X-100, 0.05% Tween® 20, 1 mg/ml Pluronic® F-127) were mixed at ambient temperature for 1 h. Under these conditions, the mixture is expected to comprise beads not bound to a DNA molecule (37%), beads bound to 1 DNA (37%) and beads bound to several DNAs (26%).

The sample containing the DNA/bead complexes (at a concentration of between 20 and 100 pM) was then injected into the observation chamber. The minimum incubation time is 3 h. Rinsing was carried out with the same solution as that used to passivate the chamber (“passivation solution”).

The results showed that the passivation step is efficient: the bead/DNA complexes were located on the anchoring protein deposits.

We also confirmed that the size of the patterns (regions) was determining for making it possible to isolate a single bead/DNA complex: for example, in FIG. 10A, it can be seen that several bead/DNA complexes attach to 5 μm-sided anchoring protein patterns (10.3 beads/pattern), whereas the patterns of 600 nm (FIG. 10C) are occupied predominantly by a single bead/DNA complex (64%) (in the two cases, the DNA molecule has a length of 2080 by and the bead a diameter of 300 nm). Moreover, with patterns of 800 nm and for DNA molecules of 798 bp, virtually all (90%) of the sites are occupied by a single bead/DNA complex valid for the analysis.

### 4. Simultaneous Monitoring of The Conformational Dynamics of a Large Number of Individual DNA Molecules by Optical Video Microscopy Coupled to Image Analysis

We subsequently placed the observation chamber on the platform of an optical microscope used in epifluorescence mode.

The presence of the beads fixed in an ordered manner to the anchoring protein patterns made it possible to rapidly bring into focus the region of interest.

The dynamic parameters of the bead present in the region of interest were then analyzed using a computer program implemented under Labview®.

This program carries out:
the registration of images of the beads over time (between 25 Hz and 1 kHz);
a preliminary thresholding necessary for determining the position of the beads. The positions of the beads in an image are calculated by taking the barycenter of the intensities of the pixels contained in 10 to 20 pixel-sided zones centered on the particles;
the calculation of the points of anchoring of the beads, by averaging the position of the beads over a period of time sufficient for the beads to have explored all of their range of freedom. The sufficient period of time is estimated at 2 seconds of acquisition at an acquisition frequency of 25 images per second (Pouget et al., 2004). The formula characterizing this calculation is of the form:

$${X}_{i - ( Nwin - 1 ) / 2} = \frac{{\sum }_{k = i - Nwin + 1}^{i} {x}_{k}}{Nwin} , \\ {Y}_{i - ( Nwin - 1 ) / 2} = \frac{{\sum }_{k = i - Nwin + 1}^{i} {y}_{k}}{Nwin} ,$$

for i=Nwin to Ntot
(X_{i-(Nwin-1)/2}, Y_{i-(Nwin-1)/2}) are the coordinates of the anchoring point

(xₖ, yₖ) are the coordinates of the center of the bead

“Nwin” is the size of the sliding window;
the calculation of the amplitude of the motion of the beads around their anchoring point, by calculating the quadratic mean of the distances of the beads to their anchoring point.

The program carries out a first operation which consists in centering the positions of the particle on the anchoring point (X_{c}, Y_{c}) by applying the following formula:

x_{c}_{_{i−(Nwin−1)/2}}=x_{i-(Nwin-1)/2}−X₀_{_{i−(Nwin−1)/2}}

and

y_{c}_{_{i−(Nwin−1)/2}}=y_{i-(Nwin-1)/2}−Y₀_{_{i−(Nwin−1)/2}}

where (X₀_{ₖ}, Y₀ₖ) are the coordinates of the first calculated anchoring point, and constitutes the reference anchoring point.

This calculation makes it possible to qualify the movement of the marker over time, relative to the current anchoring point, and the formula used is the following:

$${Aeq}_{i - ( Nwm - 1 )} = \sqrt{\frac{1}{Nwin} * {\sum }_{k = i - \frac{( Nwin - 1 )}{2} - ( Nwin - 1 )}^{i - \frac{( Nwin - 1 )}{2}} ( {x}_{{c}_{k}}^{2} + {y}_{{c}_{k}}^{2} )} ,$$

for i=2Nwin−1 to Ntot Aeq_{i-(Nwin-1)} is the amplitude of the motion of the marker at the iteration i−(Nwin−1);
calculations to verify the validity of the motion of the bead, for example the calculation of the motion of the anchoring point and the bead motion asymmetry factor.

The program calculates the motion of the anchoring point using the formula:

$${amplAnc}_{i - ( Nwin - 1 )} = \sqrt{\frac{1}{Nwin} * {\sum }_{\begin{matrix}k - i - \frac{( Nwin - 1 )}{2} - ( Nwin - 1 ) \\ ( Nwin - 1 )\end{matrix}}^{i - \frac{( Nwin - 1 )}{2}} ( {X}_{k}^{2} + {Y}_{k}^{2} ) ,}$$

amp/Anc_{i-(Nwin-1)} is the amplitude of the motion of the anchoring point at the iteration i

(Xₖ, Yₖ) are the coordinates of the anchoring point at the iteration k.

These calculations make it possible to verify that the bead or a part of the DNA is not nonspecifically adsorbed onto the surface (visible by an abrupt movement of the anchoring point).

The asymmetry factor characterizes the circularity of the distribution of the bead positions.

Firstly, it is necessary to construct the matrix of covariance C such that:

$$C = [ \begin{matrix}{σ}_{xx} & {σ}_{xy} \\ {σ}_{xy} & {σ}_{yy}\end{matrix} ] , \\ and$$

$${σ}_{xx} = \frac{1}{N} {\sum }_{i = 1}^{N} {x}_{i}^{2} - \frac{1}{{N}^{2}} {( {\sum }_{i = 1}^{N} {x}_{i} )}^{2}$$

$${σ}_{xy} = \frac{1}{N} {\sum }_{i = 1}^{N} {x}_{i} * {y}_{i} - \frac{1}{{N}^{2}} ( {\sum }_{i = 1}^{N} {x}_{i} ) * ( {\sum }_{i = 1}^{N} {x}_{i} )$$

“N” is the size of the sliding window,

(xi , yᵢ) are the coordinates of the center of the marker at the iteration i.

Secondly, the program calculates the actual values of the matrix of covariance C: λ1 and

$$\sqrt{\frac{{λ}_{max}}{{λ}_{min}}}$$

λ2. These values have a ratio proportional to the eccentricity of the ellipse in which the positions of the bead lie. For an asymmetry factor equal to 1, the entire distribution is included in a circle.

These calculations make it possible to verify that the bead is attached to the support only via a single DNA.

The first measurements of dynamic parameters of the DNA-coupled beads located on anchoring protein patterns created by molecular stamping were carried out with a DNA of 2080 by and a bead 300 nm in diameter.

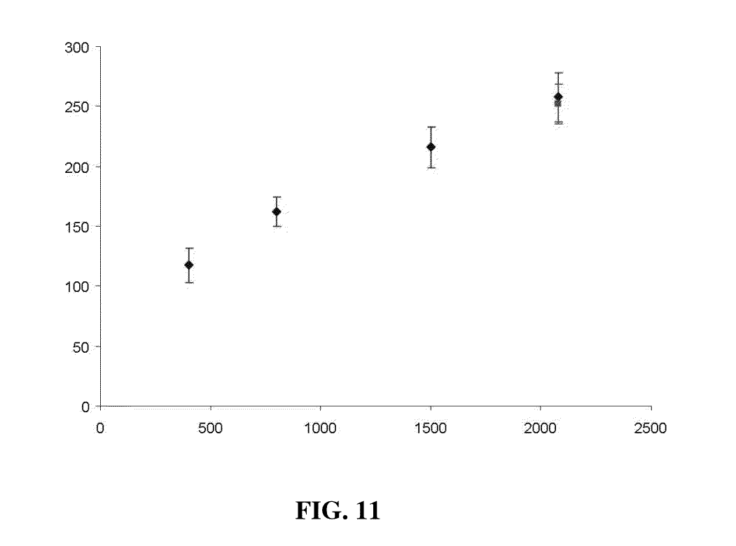

The measured mean value of the amplitude of motion of this DNA/bead couple is 252.0±16.4 nm. This value is in agreement with the value measured for this same DNA/bead couple under “conventional” TPM conditions (257.8±20.6 nm). We therefore extrapolated the values that we will obtain with shorter DNAs on these same structured media and constructed a calibration curve for this measurement technique (see FIG. 11).

### 5. Analysis of the Activity of a Nucleo-Enzyme Using a Biochip According to the Invention

In order to demonstrate the informative potential of the biochips according to the invention, we analyzed the rate of degradation by the T7 bacteriophage exonuclease, never yet studied as a single molecule, on DNA molecules of 2080 bp.

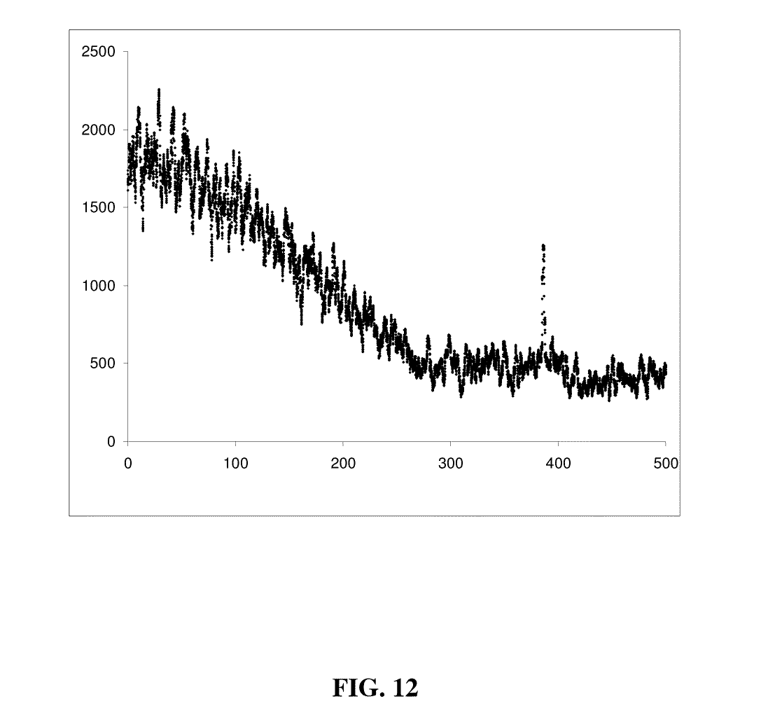

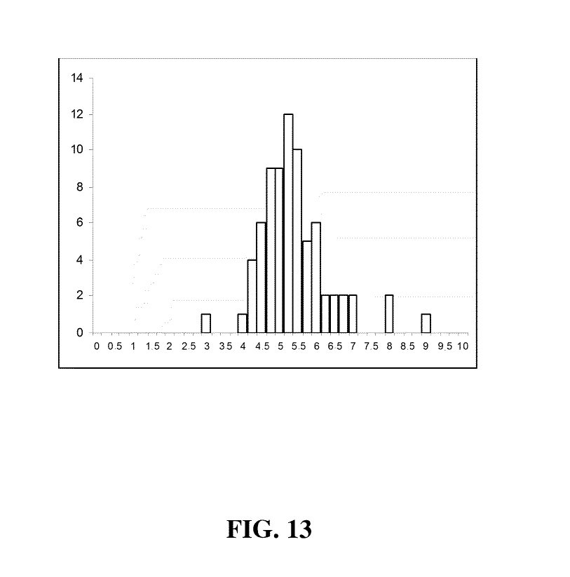

In order to create a specific binding site for the exonuclease on the double-stranded DNA molecules, we used the endonuclease Nb.BbvCI (New England Biolabs) which cleaves one of the strands at a distance of 500 nucleotides from the biotinylated nucleotide (5 units of enzyme for 40 ng of DNA, step in solution in a 10 mM Tris-HCl buffer, containing 50 mM NaCl, 10 mM MgCl₂, 1 mM dithiothreitol, 0.1 mg/ml BSA, pH 7.9). The DNA molecules are then coupled to the beads according to the protocol previously described, and then the bead/DNA complexes are introduced into the observation chamber. The exonuclease (5 units, in a 10 mM Tris-HCl buffer containing 50 mM NaCl, 10 mM MgCl ₂, 1 mM dithiothreitol, 0.1 mg/ml BSA, 1 mg/ml Pluronic® F-127, pH 7.9) is then injected into the chamber and the trajectories of 120 beads were simultaneously recorded over time. A decrease in the amplitude of the motion is observed, corresponding to the gradual degradation of the double-stranded DNA to single-stranded DNA very probably from the specific site of binding of the enzyme (see FIG. 12). The parallelized acquisition of a large number of trajectories made it possible to directly construct the histogram of the rates of degradation by the exonuclease (FIG. 13).

1. A biochip comprising a substrate, said substrate comprising at its surface isolated regions for the anchoring of a nucleic acid molecule, said isolated regions having an area of less than 1 μm², and the space between two isolated regions being at least equal to the square root of the value of said area of said isolated regions.

2. The biochip as claimed in claim 1, characterized in that said isolated regions have a layer of molecules for the anchoring of a nucleic acid molecule.

3. The biochip as claimed in claim 2, characterized in that said molecules for the anchoring of a nucleic acid molecule are chosen from streptavidin; avidin; streptavidin derivatives and avidin derivatives, in particular neutravidin; antibodies, in particular anti-digoxigenin, anti-BSA and anti-carboxyfluorescein antibodies; oligonucleotides or functionalized oligonucleotides.

4. The biochip as claimed in claim 1, characterized in that each isolated region enables the anchoring only of a single nucleic acid molecule.

5. The biochip as claimed in claim 1, characterized in that said substrate is chosen from an inorganic substrate; an organic substrate, in particular a polymer substrate; and a metal substrate.

6. The biochip as claimed in claim 1, characterized in that said substrate is a glass coverslip functionalized with epoxides, or a glass coverslip functionalized with a PEG/PEG-biotin mixture.

7. The biochip as claimed in claim 1, characterized in that said isolated regions have an area of less than or equal to 0.9 μm², 0.8 μm², 0.7 μm², 0.6 μm², 0.5 μm², 0.4 μm², 0.3 μm², 0.2 μm², 0.1 μm², 0.09 μm², 0.07 m², 0.05 μm² or 0.04 μm².

8. The biochip as claimed in claim 1, characterized in that said isolated regions have a square shape with a side of less than 1 μm, particularly less than or equal to 900 nm, 800 nm, 700 nm, 600 nm, 500 nm, 400 nm, 300 nm, or more particularly less than or equal to 200 nm.

9. The biochip as claimed in claim 1, characterized in that each isolated region enables the anchoring of a single nucleic acid molecule, the characteristic dimension of said nucleic acid molecule being greater than half the square root of the value of the area of the isolated region on which said nucleic acid molecule is attached.

10. The biochip as claimed in claim 1, characterized in that it also comprises a coverslip positioned above the substrate, said coverslip comprising at least two openings for the introduction of solutions at the level of the isolated regions, the whole assembly defining an observation chamber.

11. A process for fabricating a biochip, comprising the following steps:
(a) providing a substrate, and
(b) printing on said substrate isolated regions for the anchoring of a nucleic acid molecule, said isolated regions having an area of less than 1 μm², and the space between two isolated regions being at least equal to the square root of the value of said area of said isolated regions.

12. The process as claimed in claim 11, characterized in that step (b) consists in printing on the substrate, in said isolated regions, a layer of molecules for the anchoring of a nucleic acid molecule.

13. The process as claimed in claim 12, characterized in that, before step (b), the substrate is treated so as to attach said molecules for the anchoring of a nucleic acid molecule.

14. The process as claimed in claim 11, characterized in that step (b) is carried out according to the microcontact printing, “lite-off” or “inverted print” method.

15. A process for studying nucleic acid molecules using the “Tethered Particle Motion” or “TPM” technique, comprising the steps of:
1) providing a biochip as defined in claim 1,
2) treating the nucleic acid molecules so as, on the one hand, to be able to attach them to the biochip and, on the other hand, to be able to analyze them using the “Tethered Particle Motion” or “TPM” technique,
3) studying the nucleic acid molecules using the “Tethered Particle Motion” or “TPM” technique.

16. The process as claimed in claim 15, characterized in that the characteristic dimension of said nucleic acid molecules is greater than half the square root of the value of the area of said isolated regions of the biochip.

17. A kit comprising:
a biochip as defined in claim 1, and
a computer-readable medium comprising instructions which can be executed by said computer in order to implement a process, the process comprising:
1) providing the biochip;
2) treating the nucleic acid molecules so as, on the one hand, to be able to attach them to the biochip and, on the other hand, to be able to analyze them using the “Tethered Particle Motion” or “TPM” technique; and
3) studying the nucleic acid molecules using the “Tethered Particle Motion” or “TPM” technique.

18. The use of a biochip as defined in claim 1 for studying nucleic acid molecules using the “Tethered Particle Motion” or “TPM” technique.

19. The use as claimed in claim 18, characterized in that the characteristic dimension of said nucleic acid molecules is greater than half the square root of the value of the area of said isolated regions of the biochip.
